(19) United States
(12) Patent Application Publication
(10) Pub. No.: US 2010/0262211 A1
(43) Pub. Date: Oct. 14, 2010

(54) Use of a Coated, Transparent Substrate for Influencing the Human Psyche

(76) Inventors: Walther Glaubitt, Margetshöchheim (DE); Jörn Probst, Kürnach (DE)

Correspondence Address: GUDRUN E. HUCKETT DRAUDT, SCHUBERTSTR. 15A, WUPPERTAL, 42289, DE

(21) Appl. No.: 12/742,532

(22) PCT Filed: Nov. 5, 2008
(86) PCT No.: PCT/EP2008/064989

§ 371 Date: May 12, 2010

(30) Foreign Application Priority Data
Nov. 12, 2007 (DE) 10 2007 053 839.3

(51) Int. Cl. A61N 5/06

(52) U.S. Cl. 607/88

(57) ABSTRACT

In order to influence the human melatonin reservoir, the invention proposes the use of bodies, particularly glasses, having a transmittivity for light waves of a wavelength of about 460 nm of at least 92%, preferably of at least 95% or even 99%, as window glazings, for example, in the construction of houses, for sunrooms, or for indoor riding arenas. If said bodies are coated bodies, the invention proposes the use of a coating sol or gel, containing a hydrolyzable or partially or completely hydrolyzed silane and/or SiO₂₋ and/or ZrO₂₋ particle for producing the coating.

The invention concerns coatings or substrates with an etching and/or a coating on the basis of in particular nano-porous SiO₂ that, with respect to their transparency, are matched to the spectral course of intensity of natural light with special consideration of the psychologically effective wavelength ranges. Potential fields of application reside in the glazing of buildings in which humans stay temporarily or permanently for the purpose of living, working, spending leisure time or therapy, and in auxiliary glazing, filters or lenses for irradiation devices, light therapy devices or illuminations for wellness.

For a long time the human eye has been viewed purely as an organ of vision. Only three and a half decades ago, the discovery of the retina-hypothalamic tract (RHT) (R. Y. Moore and N. J. Lenn, J. Comp. Neurol., 1972, 146, 1-14) provided evidence that a direct nerve connection between the retina and the hypothalamus exists. At one end thereof the so-called NIF (non image forming) receptors are located in the retina whose spectral sensitivity is in the range of 380 nm and 580 nm (G. C. Brainard, J. Neuroscience 21, 2001, 16, 6405-12; K. Thapan, J. Physiology, 2001, 1, 261-7; D. Gall, LICHT 54, 2002, 11-12, 1292-7). They serve for conducting light/dark signals of visual stimulation to the suprachiasmatic nucleus (SCN) at the other end of the RHT located directly above the optic chiasma. The SCN is viewed as the anatomical seat of the biological clock. The stimulations received thereat have an effect on numerous vegetative and hormonal functions in the human body, inter alia, the melatonin balance important for the sleep/wake cycle.

When the NIF receptors receive radiation of an intensity that is too minimal in the corresponding wavelength range, this can lead to a disruption of the melatonin balance which has a detrimental effect on mental/psychic well-being of the human. Possible effects of an under-supply are sleep disturbances, depressions or other psychic illnesses. This relation becomes especially apparent in the examination of the phenomenon of “winter depression” which is indeed diagnosed frequently in light-poor winter months. According to statistics of the Department of Labor in North Rhine-Westphalia 27% of all records of occupational disability are based on psychic illnesses of which a major proportion is assigned causatively to the melatonin control mechanism.

Past developments of glazing have concentrated exclusively on optimizing antireflective action in the range of maximum light sensitivity of the human retina (approximately 555 nm at day light) resulting in transmittivities above 96%. Main fields of applications are transparent display windows, facades, lobby areas, and observation rooms with large light differences before and behind glass. Examples of products used in this sector of the market are AMIRAN® of the Schott company and CENTROSOL structure glass of the Centrosolar company.

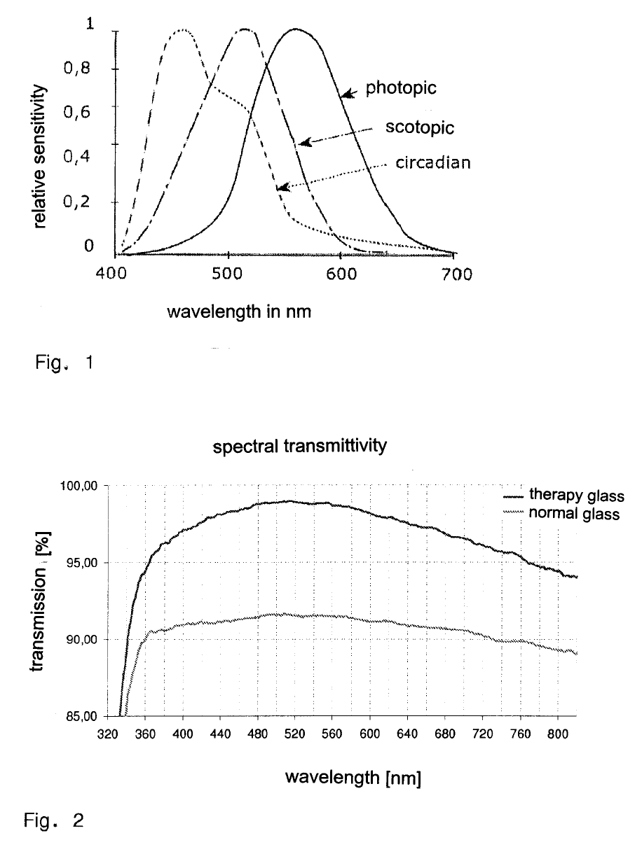

In this connection, the health effects of optical radiation on the human organism are not taken into consideration in this connection, in particular, the photoinduced melatonin suppression, a circadian (from Latin circa: about; dia: day) based process in the human organism that controls the inner clock and whose disturbances lead to various disturbances of body functions. Brainard (see I.c.) and Thapan (see I.c.) have found that the relative spectral efficacy of the melatonin suppression in comparison to the light intensity curve for vision during the day is displaced toward the shortwave range of the visible spectrum. This is illustrated in FIG. 1 showing the sensitivity curves of the receptors in the eye. The solid line curve positioned farthest to the right with the maximum at approximately 560 nm represents the spectral sensitivity of the light intensity of the human eye which corresponds to the sensitivity of the rod receptors in the eye. It represents thus the photometric sensitivity and is characterized as the photopic curve. The middle dashed line curve represents the spectral sensitivity of the rod receptors of the human eye and thus the sensitivity for night vision; it is referred to as scotopic curve. All the way to the left, with the maximum at the shortest wavelength, there is a dash-dotted line that has been determined empirically for the receptors that control the melatonin suppression (circadian curve). Based on this, it is apparent that the blue components of the light are more effective with respect to melatonin suppression, with a maximum of efficacy at around 460 nm.

It is the object of the invention to provide glass or other bodies that are transparent for day light that take into consideration this situation and are modified such that they prevent a possible winter depression or other negative effects of the human melatonin balance or ameliorate them.

The object of the invention is solved by the proposal to provide for this application glass or other preferably flat bodies that are transmissive for day light, that are designed such that in the wavelength range of approximately 460 nm they have a high transmittivity that is at least approximately 92%, preferably at least 95%, and especially preferred at least 98%. Preferably, in this connection glasses with coatings are used that develop their transmittivity in the range of 450 nm to 550 nm in order to combine both aspects, i.e., a glass that is invisible as much as possible because it is reflection-free and a yield as high as possible of circadian-effective radiation proportions of the light source.

At perpendicular impact of light at the boundaries surfaces of air to glass reflection losses of 4% result. Together with further losses by absorption in the glass conventional glasses such as soda lime glasses have therefore an average visual transmission of approximately 91%. Conventional industrial methods for antireflective properties of glass utilize the interference principle. In this connection, alternatingly two or more layers of high-refractive or low-refractive materials are stacked on top one another. In a certain wavelength range the waves reflected at the boundary surface will cancel one another. The effect is reversed to an increased reflection at wavelengths that have double the size of the design wavelengths. Therefore, the band width of anti-reflection is limited to a maximum of one octave and is not suitable for anti-reflective action of the solar spectrum with broader band width. This limitation can however be circumvented by means of a physical concept that has been known for a long time and is also based on the interference principle but enables the required extremely low refractive indices in that a coating (or uppermost layer) is provided whose (coating) material is diluted with air. For an optimal antireflective action basically only two conditions must be fulfilled in order to achieve a complete destructive interference in air. The first is the phase condition; it is:

λ(nm)=4×nₛ×Dₛ (1)

wherein
λ=wavelength
nₛ=refractive index of the layer
Dₛ=thickness of the layer

The second is the amplitude condition; it is:

nₛ=\sqrt{n}_{G} (2)

with n_{G}=refractive index of the glass on which the layer is located (the refractive index of air is 1).

When window glass with a refractive index of 1.51 is used, the optimal refractive index of the layer is 1.23. In order to achieve optimal anti-reflection at 460 nm, the layer with this refractive index must have a thickness of

460 nm:1.23×4=94 nm.

Such layer thickness is achieved, for example, in that the substrate to be coated is immersed in an immersion bath of suitable coating materials, for example, sols, pulled out at an appropriate drawing speed, and subsequently dried or heated. The precise drawing speed is empirically determined in a beneficial way based on a calibration curve: The faster the drawing action, the thicker the layer. Alternatively, the layer can be produced by etching the glass.

When however the refractive index of the porous layer is not optimal, the layer thickness must be adjusted appropriately. When the refractive index is, for example, 1.32, a reflection minimum at 460 nm results when the layer has a thickness of only 87 nm. Still, the reflection minimum of this layer is of course less than optimal. The residual reflection according to the Fresnel equation is:

$$R = {( {1.32}^{2} - \frac{1.51}{{1.32}^{2}} + 1.51 )}^{2} \times 100 \%$$

i.e., 0.5%.

With these basic principles, it can be easily determined which glasses with which coating/etching are usable for the present invention.

The development of single layers on glass that have the low refractive index required in accordance with the invention already started in the 40s of the last century. The methods described since can be divided into three areas. The first concerns the direct etching of glass, the second the porous coatings, and the third is a combination of both. Here, the layers having too low a porosity are subsequently etched.

Porous layers that are produced by etching of glass are characterized by excellent optical results (see Soren Milton Thompson, Verfahren zur Herstellung eines die Reflexion vermindernden Films auf der Oberfläche eines Glasgegenstandes, DE patent 822714, 1949; M. J. Minot, Single-layer, Gradient Refractive Index AR films Effective from 0.35 to 2.5 μm, J. Opt. Soc. Am. 66, (1976) 515; and G. K. Chinyama, A. Roos, and B. Karlson, Stability of Antireflection Coatings for Large Area Glazings, Solar Energy 50, (1993) 105). Layers of soda lime glass produced in this way achieve a reflective index of 1.27 (Wagner, A., Industrielle Fertigung von Solar-Antireflexglas, 11. Symposium Thermische Solarenergie, Ostbayerisches Technologie-Transfer-Institut e.V., Kloster Banz, 9-11 May 2001). When such an etching layer is applied at a depth of approximately 100 to 130 nm, the soda lime glasses treated in this way are suitable for the purposes of the present invention. A further etching method may be used for glasses that undergo phase separation, for example, borosilicate glass of the composition 55-82% SiO₂, 12-30% B₂O₃, 2-12% alkali metal oxides and 0-7% Al₂O₃ (values in weight %) (J. A. Doddato, M. J. Minot, Durable Substrates Having Porous Antireflection Coatings, U.S. Pat. No. 4,080,188 (1978). This also leads for correspondingly thick etching layers to transmittivities that are suitable according to the invention even when the complex etching method and the use of dangerous acids such as half-concentrated hexafluoro silicic acid or NH₄F—HF is disadvantageous in this connection (Nostell, P., Roos, A.; Karlsson, B.; Antireflection of glazings for solar energy applications, Solar Energy Materials and Solar Cells 54, (1998) 223-233).

Suitable coating solution for porous layers have been found by Moulten already in the year 1943 (H. R. Moulton, Method of producing thin microporous silica coatings having reflection reducing characteristics and the articles so coated, U.S. Pat. No. 2,474,061 (1949)). He employed mixtures of tetraalkoxy silane, ethyl acetate, ethanol, water with HCl and produced therefrom sols with which glass panes were coated. The porous layers created after a thermal treatment on glass having a refractive index of 1.52 raised the transmission for the design wavelength to 98% so that glasses coated in this way are usable for the present invention.

1983 Yoldas (B. E. Yoldas, Antireflective Graded Index Silica Coating, Method for Making, U.S. Pat. No. 4,535,026) further improved the transmission to 99.5% by using polished silica glass and this across a wavelength range of 300 nm to 1,100 nm. The applied porous layer according to Moulten was etched in this connections so that the pore volume was increased and therefore the refractive index was lowered. At the same time, by means of etching a pore radius gradient resulted that led to broadening of the reflection minimum. Accordingly, the aforementioned silica glass is especially well-suited for the present invention.

For equipping solar collectors, porous layers with a refractive index of 1.27 were produced at 500° C. by using sodium silicate particles of a size of 25 nm (K. J. Cathro, D. C. Constable, and T. Solaga, Silica Low-Reflection Coatings for Collector Covers, By a Dip-Coating Process, Solar Energy 32, (1984) 573), and it was proposed to lower the refractive index by etching the porous layer to the optimal value (R. B. Pettit, C. S. Ashley, S. T. Reed, C. J. Brinker, Antireflective Films from the Sol-Gel Process, in: Sol-Gel Technology for Thin Films, Fibers, Preforms, Electronics, and Specialty Shapes, edited by Lisa C. Klein, Noyes Publications, New Jersey, USA, 1988, pp. 81-109) In addition to the pure SiO₂ systems, porous layers have been developed also which chemically are similar to the composition of borosilicate glass. A disadvantage in this connection is also that the layers of unsatisfactory porosity must be etched in order to achieve a high solar transmission (C. S. Ashley, S. T. Reed, Sol Gel AR Films for Solar Application, Mat. Res. Soc. Symp. Proc., 73, 671-677). In this way, an average solar transmission between 95.6% and 96.8% was achieved, compared to 92% for the uncoated glass (R. B. Pettit and C J. Brinker, Use of sol-gel thin films in solar energy applications, Solar Energy Mater. 14 (1986) 269-28).

Yoldas (see I.c.) and later on Vong (M. S. W. Vong and P. A. Sermon, Observing the breathing of silica sol-gel derived anti-reflection optical coatings, Thin Solid Films 293, (1997) 185) have noted that at temperatures above 400° C. the porous SiO₂ structure begins to sinter wherein the already achieved pore volume decreases again in conjunction with an undesirable increase of the refractive index. This effect has also been disclosed by Takamatsu et al. (Takamatsu, Atsushi, Refectance reducing film and method of forming same on glass substrate, EP 0 597 490 A1). They achieve a residual reflection of only 1.2% at 550 nm, after the coated glass has been exposed for 10 minutes to 550° C. A temperature treatment at 600° C. further compacts the layer and increases the residual reflection to 3%. For the manufacture of scratch-resistant anti-reflection layers this is very problematic because sufficiently stable scratch-resistant porous layers on glass were obtained only at temperatures of at least 500° C. (see Cathro et al., I.c.), with even better results in the softening range of the glass (see H. R. Moulton, Composition for Reduction the Reflection of Light, U.S. Pat. No. 2,601,123).

It has therefore been attempted to develop porous and sinter-stable SiO₂ layers. For this purpose, at Central Glass Company, Japan, tetraethoxy silane was hydrolyzed and condensed in the presence of acid and organic polymers, for example, polyvinyl acetate, with an average molecular weight of 83,000 g/mol. Before the coated glass was heated to temperatures between 570° C. to 670° C., the organic polymer within the layer was extracted by an alcohol-water mixture. After tempering, a scratch-resistant porous SiO₂ layer was obtained (Yamazaki, Seiji, Porous Metal-Oxide Thin Film and Method of Forming Same On Glass Substrate, WO 97/06896). However, there are no data in regard to obtained antireflective action or refractive index in the description or the claims so that one can only assume that an undesirable compaction may have occurred. Sinter-stable anti-reflection (AR) layers are however actually known. They have been disclosed by Glaubitt et al. (Glaubitt, W; Becker, H.; Vorgespanntes, mit einer wischfesten, porösen SiO₂-Antireflex-Schicht versehenes Sicherheitsglas und Verfahren zu dessen Herstellung, DE 199 18 811 A1). It has been found that in case of porous SiO₂ layers, that have been by a method developed also by Glaubitt et al. (Glaubitt, W.; Gombert, A.; Verfahren und Beschichtungszusammensetzung zur Herstellung einer Antireflexionsbeschichtung, DE 196 42 419 AI), even after 15-minute exposure at temperatures of 800° C. up to 1,000° C. and even somewhat above, no significant compaction happens and in this way a residual reflection at 460 nm of between approximately 0.7% and 4.0% can be obtained. In this way, it is possible to produce at temperatures of more than 650° C. prestressed safety glass that is furnished with a porous anti-reflection layer with almost optimal efficiency. By testing the layer according to DIN EN 1096-2, according to which a rotating abrasion finger of metal that is coated with felt is moved forward and back across the coated glass plates at a load of 4 N, a moderate wear resistance was confirmed (10 strokes).

There is no lack of attempts to provide scratch-resistant layers even at low temperatures. Thomas (I. M. Thomas, Method for the preparation of porous silica antireflection coatings varying in refractive index from 1.22 to 1.44, Appl. Opt. 31, (1992) 6145) examined in this connection a mixture of silicate particles in a molecular siloxane matrix that has been developed also substantially by Moulton (U.S. Pat. No. 2,601,123) and found that the layers produced at low temperatures became sufficiently scratch-resistant only when the pore volume in the layer dropped and the refractive index increased. Floch (H. G. Floch and P. F. Belleville, A Scratch-Resistant Single-Layer Antireflective Coating by a Low Temperature Sol-Gel Route, J. Sol-Gel Sei. Tech. 1, (1994) 293-304; H. G. Floch and P. F. Belleville, Damage-Resistant Sol-Gel Optical Coatings for Advanced Lasers at CEL-V, J. Sol-Gel Sci. Tech. 2, (1994) 695-705) increased the scratch resistance of the layer in that the molecular siloxane that functioned as a binder between the particles in Thomas was replaced by a derivative of polytetrafluoroethylene without however being able to prevent an increase of the refractive index.

All of the afore described sols are alcohol-water mixtures, i.e., partially aqueous systems. The use of aqueous oxidic sols (SiO₂/ZrO₂ sols) that have less than 1% organic components for such coatings has also been disclosed (see, for example, Glaubitt, W., Schulz, J., Dislich, H., König, F., Büttgenbach, L., Verfahren zur Abscheidung poröser optischer Schichten, DE 198 28 231 C2). The layer thickness can be adjusted for a single coating to 30 to 300 nm and thus to the optimized thickness for the present invention. Such sols that contain surface-active ingredients and are practically purely aqueous increase the solar transmission of an iron-poor soda lime glass equipped therewith to 95.3% wherein the layer has a refractive index of 1.29. Such layers do not achieve the optimal refractive index but they are extremely scratch-resistant (1,000 strokes).

Glaubitt et al. (Glaubitt, W., Kursawe, M., Gombert, A, Hofmann, Th., Neuartiges Hybridsol zur Herstellung abriebfester SiO₂-Antireflexschichten, WO 03/027015 A1) have found that, when using silicate particles that are dispersed in water and have been added to the instable ammoniacal sols, the scratch resistance of the resulting layer is drastically increased without being compacted by an appreciable amount during the thermal loading at temperatures of 650° C. The residual reflection is at ≦0.5% and 1,000 strokes according to DIN EN 1096-2 were measured. However, this is successful only when the ammoniacal sols have reached a certain age, i.e., they themselves already have generated particles before they are added to the aqueous silica sol. When doing so, the thus-produced mixtures will heat up, interestingly enough also those to which already more water has been added than would have been required for a complete hydrolysis of the tetraalkoxy silane (>4 mol/mol). The heat indicates a reaction in the presence of and with possible participation of the added particles.

All coating solutions according to the aforementioned methods are suitable for producing a broad-band antireflective glass with a transmission maximum in the range of 450 nm-550 nm. The desired transmission maximum can be adjusted with the aforementioned immersion processes as disclosed above by the drawing speed upon pulling out the glass out of the coating solution. But also the coatings or etchings that have been developed already earlier can lead to glasses with the desired properties and can thus be used for the purposes of the invention even though downsides must be accepted or the methods for their manufacture have disadvantages.

### EMBODIMENT ACCORDING TO WO 03/027015 A1

0.1 n ammonium hydroxide solution in a quantity of 1,994 g is completely mixed with 25,670 g of ethanol and to this, with further stirring, 3,405 g tetramethoxy silane are added.

After stirring for 2 hours, 26,880 g of aqueous 2% silica sol is added and stirring is continued for 30 minutes until also 86,925 g of 1-methoxy-2-propanol are added to the mixture. The aqueous 2% silica sol is produced according to U.S. Pat. No. 4,775,520. The coating solution is stirred over night and subsequently filtered.

Into the coating solution a previously cleaned pane of glass is immersed and is pulled out at a speed of 15 cm/min. After 10 min of venting the pane of glass is tempered at 550° C. for 15 min. A therapy glass with broad band antireflective action useable for the present invention is produced with a transmission maximum of 510 nm. See in this connection also FIG. 2 in which the spectral transmittivity of the therapy glass of this example is illustrated with a transmission maximum of approximately 99% at 510 nm in comparison to the transmittivity of normal glass (broad maximum but transmittivity hardly above 90-91%).

### EMBODIMENT ACCORDING TO DE 196 42 419 A1

7.6 g of polyethylene glycol with an average mole mass of 10,000 are dissolved in 9.5 g ammoniacal water with a pH value of 9.5 in the presence of 27.0 g methanol. This solution is added to a mixture of 15.2 g tetramethoxy silane and 80.0 g methanol. After stirring for 10 minutes the resulting mixture is filtered. After an aging period of approximately 80 minutes glass panes are coated by immersion. For obtaining an especially uniform layer of approximately 100 nm, the pane to be coated is fixed in a coating bath and the coating solution contained therein is removed within two minutes free of shocks. After the coating process the panes are dried for 30 minutes at 130° C., subsequently heated at a heating rate of 120 K/h to 500° C. and kept for one hour at this temperature. The resulting antireflective coating shows a refractive index of 1.22.

### EMBODIMENT ACCORDING TO AND DE 199 18 811 A1

First, a coating solution according to the embodiment of DE 196 42 419 A1 is produced. Into this solution sequentially eight silica glass panes are immersed and pulled out at a constant speed. Subsequently, these eight panes are each exposed for 15 minutes to different temperatures between 500 and 1,200° C. In samples exposed to temperatures between 600° C. and 1,000° C. at 460 nm reflections of approximately 5.5% to below 2% (reduced reflection at higher temperature) are measured. When the sample is exposed to 1,100° C., the reflection at 460 nm drops even significantly below 1%.

What is claimed is:

1.-18. (canceled)

19. A method of influencing in a human being the human melatonin balance, the method comprising the steps of providing a light-transmissive body with a transmittivity of at least 92% for light waves of a wavelength of approximately 460 nm and exposing the human being to light passing through said light-transmissive body.

20. The method according to claim 19 for ameliorating winter depression or sleep disturbances.

21. The method according to claim 19, wherein said light-transmissive body has a transmittivity for said light waves of at least 95%, preferably of at least 98%, and especially preferred of at least 99%.

22. The method according to claim 19, wherein said transmittivity exists across a wavelength range of 450 to 550 nm.

23. The method according to claim 19, wherein said light-transmissive body is a glass used in construction, in particular as window panes, as glazing of a sunroom, as glazing for an equestrian arena for therapeutic riding or as a lamp glazing.

24. The method according to claim 23, wherein said light-transmissive body is selected from an etched glass or a coated glass or a glass that is coated and etched.

25. The method according to claim 23, wherein said glass is a white glass or a green glass, in particular a soda lime glass that optionally contains further additives.

26. The method according to claim 19, wherein said light-transmissive body is scratch-resistant and/or a safety glass.

27. The method according to claim 19, wherein said light-transmissive body is a glass with a coating of a nano-porous oxide or a nano-porous oxide mixture.

28. The method according to claim 27, wherein said oxide is SiO₂ and wherein said oxide mixture is SiO₂ in mixture with one or several further metal oxides, in particular ZrO₂.

29. The method according to claim 28, wherein said coating is produced by applying an aqueous sol, containing SiO₂ or starting materials that are convertible by hydrolytic condensation into SiO₂ and optionally containing an organic component, onto said light-transmissive body, transferring the sol into a gel, and drying/sintering said coating.

30. The method according to claim 28, wherein said coating is generated by applying a composition, produced by adding silicate particles dispersed in water to an instable ammonia cal sol, onto said light-transmissive body, transferring the sol into a gel, and drying/sintering said coating.

31. A method of influencing in a human being the human melatonin balance, the method comprising the steps of:
providing a coating sol or coating gel, containing a hydrolyzable or partially or completely hydrolyzed silane and/or SiO₂ particles and/or ZrO₂ particles for producing a coated light-transmissive body that after coating has a transmittivity of at least 92% for light waves of a wavelength of approximately 460 nm; and
exposing the human being to light passing through said coated light-transmissive body.

32. The method according to claim 31 for improving the mental and psychic well-being of the human being especially by ameliorating winter depression or sleep disturbances.

33. The method according to claim 31, wherein said coated light-transmissive body has a transmittivity for said light waves of at least 95%, preferably of at least 98% and especially preferred of at least 99%.

34. The method according to claim 31, wherein said transmittivity exists across a wavelength range of 450 to 550 nm.

35. The method according to claim 31, wherein said coated light-transmissive body is a glass that is used in construction, in particular as window panes, as glazing of a sunroom, as glazing for an equestrian arena for therapeutic riding or as a lamp glazing.

36. The method according to claim 35, wherein said glass is a white glass or a green glass, in particular a soda lime glass that contains optionally further additives.

37. The method according to claim 31, wherein said coated light-transmissive body is scratch-resistant and/or a safety glass.
